(19) United States
(12) Patent Application Publication
(10) Pub. No.: US 2017/0361110 A1
(43) Pub. Date: Dec. 21, 2017

(54) Multi-Conductor Cable in an External Charger for an Implantable Medical Device

(71) Applicant: Boston Scientific Neuromodulation Corporation, Valencia, CA (US)

(72) Inventor: Thomas W. Stouffer, Chatsworth, CA (US)

(21) Appl. No.: 15/616,335

(22) Filed: Jun. 7, 2017

Related U.S. Application Data
(60) Provisional application No. 62/351,198, filed on Jun. 16, 2016.

(51) Int. Cl. A61N 1/378; H01F 38/14; H02J 50/90; A61N 1/36; H02J 7/02; A61N 1/372; A61N 1/05

(52) U.S. Cl. CPC A61N 1/3787; H02J 7/027; H02J 7/025; H01F 38/14; A61N 1/37229; H02J 50/90; A61N 1/36125; A61N 1/36142; A61N 1/0534; A61N 1/0551

(57) ABSTRACT

A charging system for an Implantable Medical Device (IMD) is disclosed. The charging system features an electronics module connected to a charging coil by a cable. The charging system can be configured with a belt or harness that holds the charging coil position to charge the IMD and also providing a user with easy access to the electronics module. Resistance in the cable between electronics module and the charging coil is minimized by using multiple, individually insulated conductors to carry AC current.

### CROSS-REFERENCE TO RELATED APPLICATIONS

This application claims priority to U.S. Provisional Patent Application Ser. No. 62/351,198, filed Jun. 16, 2016, which is incorporated herein by reference in its entirety.

### FIELD OF THE INVENTION

The present invention relates to wireless external chargers for use in implantable medical device systems.

### BACKGROUND

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device system.

As shown in FIGS. 1A-1C, a SCS system typically includes an Implantable Pulse Generator (IPG) 10 (Implantable Medical Device (IMD) 10 more generally), which includes a biocompatible device case 12 formed of a conductive material such as titanium for example. The case 12 typically holds the circuitry and battery 14 (FIG. 1C) necessary for the IMD 10 to function, although IMDs can also be powered via external RF energy and without a battery. The IMD 10 is coupled to electrodes 16 via one or more electrode leads 18, such that the electrodes 16 form an electrode array 20. The electrodes 16 are carried on a flexible body 22, which also houses the individual signal wires 24 coupled to each electrode. In the illustrated embodiment, there are eight electrodes (Ex) on each lead 18, although the number of leads and electrodes is application specific and therefore can vary. The leads 18 couple to the IMD 10 using lead connectors 26, which are fixed in a non-conductive header material 28, which can comprise an epoxy for example.

As shown in the cross-section of FIG. 1C, the IMD 10 typically includes a printed circuit board (PCB) 30, along with various electronic components 32 mounted to the PCB 30, some of which are discussed subsequently. Two coils (more generally, antennas) are show in the IMD 10: a telemetry coil 34 used to transmit/receive data to/from an external controller (not shown); and a charging coil 36 for charging or recharging the IMD's battery 14 using an external charger, which is discussed in detail later.

FIG. 2 shows the IMD 10 in communication with an external charger 50 used to wirelessly convey power to the IMD 10, which power can be used to recharge the IMD's battery 14. The transfer of power from the external charger 50 is enabled by a primary charging coil 52. The external charger 50, like the IMD 10, also contains a PCB 54 on which electronic components 56 are placed. Again, some of these electronic components 56 are discussed subsequently. A user interface 58, including touchable buttons and perhaps a display and a speaker, allows a patient or clinician to operate the external charger 50. A battery 60 provides power for the external charger 50, which battery 60 may itself be rechargeable. The external charger 50 can also receive AC power from a wall plug. A hand-holdable housing 62 sized to fit a user's hand contains all of the components.

Power transmission from the external charger 50 to the IMD 10 occurs wirelessly and transcutaneously through a patient's tissue 25, via inductive coupling. FIG. 3 shows details of the circuitry used to implement such functionality. Primary charging coil 52 in the external charger 50 is energized via charging circuit 64 with an AC current, Icharge, to create an AC magnetic charging field 66. This magnetic field 66 induces a current in the secondary charging coil 36 within the IMD 10, providing a voltage across coil 36 that is rectified (38) to DC levels and used to recharge the battery 14, perhaps via a battery charging and protection circuitry 40 as shown. The frequency of the magnetic field 66 can be perhaps 80 kHz or so. When charging the battery 14 in this manner, is it typical that the housing 62 of the external charger 50 touches the patient's tissue 25, perhaps with a charger holding device or the patient's clothing intervening, although this is not strictly necessary.

The IMD 10 can also communicate data back to the external charger 50 during charging using reflected impedance modulation, which is sometimes known in the art as Load Shift Keying (LSK). This involves modulating the impedance of the charging coil 36 with data bits (“LSK data”) provided by the IMD 10's control circuitry 42 to be serially transmitted from the IMD 10 to the external charger 50. For example, and depending on the logic state of a bit to be transmitted, the ends of the coil 36 can be selectively shorted to ground via transistors 44, or a transistor 46 in series with the coil 36 can be selectively open circuited, to modulate the coil 36's impedance. At the external charger 50, an LSK demodulator 68 determines whether a logic ‘0’ or ‘1’ has been transmitted by assessing the magnitude of AC voltage Vcoil that develops across the external charger's coil 52 in response to the charging current Icharge and the transmitted data, which data is then reported to the external charger's control circuitry 72 for analysis. Such back telemetry from the IMD 10 can provide useful data concerning charging to the external charger 50, such as the capacity of the IMD's battery 14, or whether charging of the battery 14 is complete and operation of the external charger 50 and the production of magnetic field 66 can cease. LSK communications are described further for example in U.S. Patent Application Publication 2013/0096652.

External charger 50 can also include one or more thermistors 71, which can be used to report the temperature (expressed as voltage Vtherm) of external charger 50 to its control circuitry 72, which can in turn control production of the magnetic field 66 such that the temperature remains within safe limits. See, e.g., U.S. Pat. No. 8,321,029, describing temperature control in an external charging device.

Vcoil across the external charger's charging coil 52 can also be assessed by alignment circuitry 70 to determine how well the external charger 50 is aligned relative to the IMD 10. This is important, because if the external charger 50 is not well aligned to the IMD 10, the magnetic field 66 produced by the charging coil 52 will not efficiently be received by the charging coil 36 in the IMD 10. Efficiency in power transmission can be quantified as the “coupling” between the transmitting coil 52 and the receiving coil 36 (k, which ranges between 0 and 1), which generally speaking comprises the extent to which power expended at the transmitting coil 52 in the external charger 50 is received at the receiving coil 36 in the IMD 10. It is generally desired that the coupling between coils 52 and 36 be as high as possible: higher coupling results in faster charging of the IMD battery 14 with the least expenditure of power in the external charger 50. Poor coupling is disfavored, as this will require high power drain (e.g., a high Icharge) in the external charger 50 to adequately charge the IMD battery 14. The use of high power depletes the battery 60 in the external charger 50, and more importantly can cause the external charger 50 to heat up, and possibly burn or injure the patient.

Charging the IMD 10 with the external charger 50 can be inconvenient if the IMD is implanted in a position that is difficult for the patient to reach. For example, an IMD used for spinal cord stimulation is typically implanted in the patient's upper buttock. A patient may have difficulty holding the external charger 50 in contact with their skin and in proper alignment for an adequate length of time to charge the battery 14. Thus, there is a need in the art for more convenient and effective methods of charging the battery of an implanted medical device.

### BRIEF DESCRIPTION OF THE DRAWINGS

FIGS. 1A-1C show different views of an implantable pulse generator, a type of implantable medical device (IMD), in accordance with the prior art.

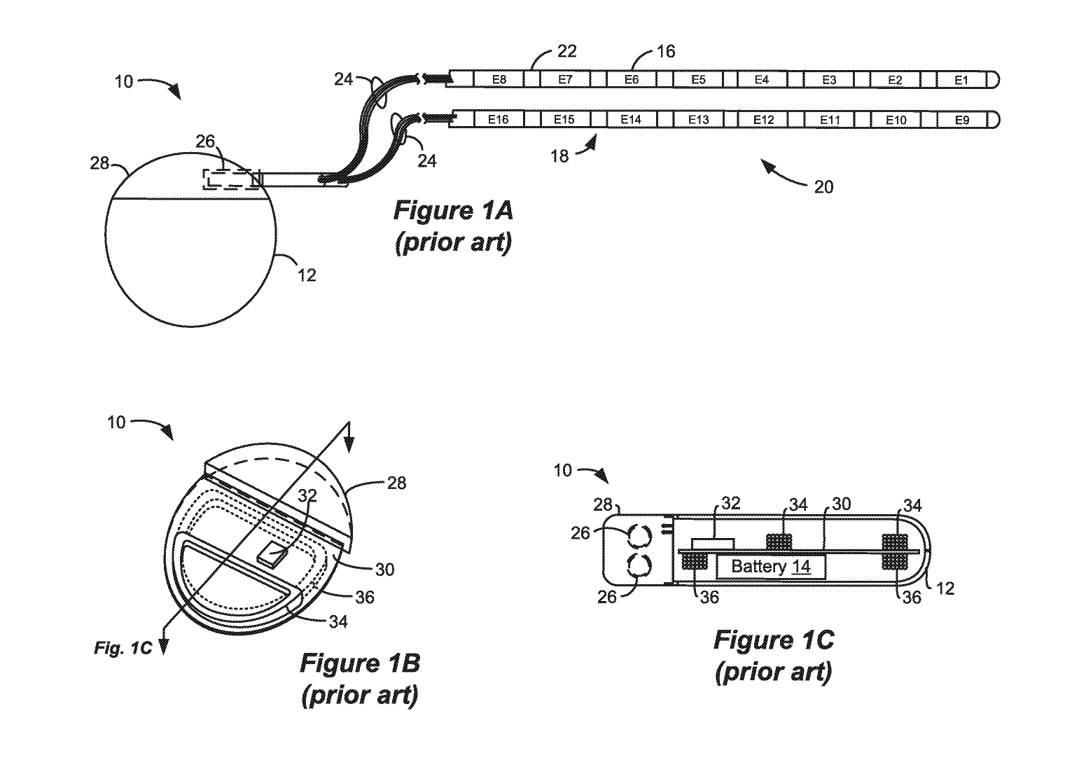

FIG. 2 shows an external charger being used to charge a battery in an IMD, while FIG. 3 shows circuitry in both, in accordance with the prior art.

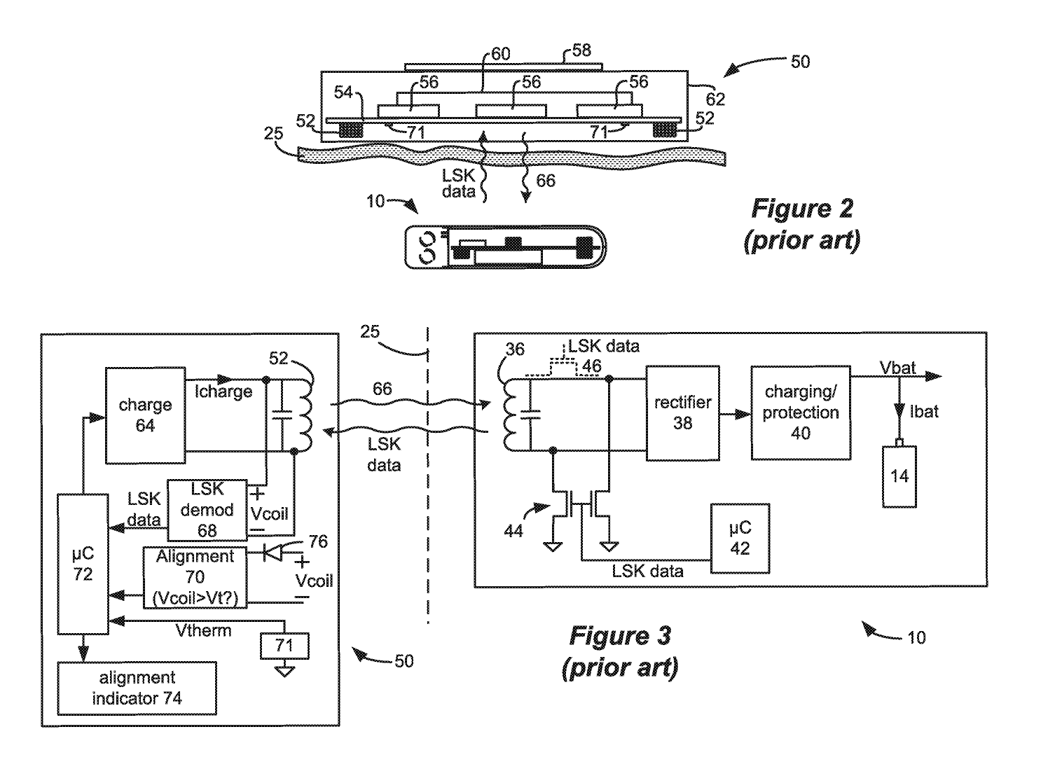

FIGS. 4A-4D show an improved charging system having a charging coil assembly and an electronics module, in accordance with an example of the invention.

FIGS. 5A and 5B show a patient wearing a carrier containing an improved charging system, in accordance with an example of the invention.

FIG. 6 shows a carrier for carrying an improved charging system, in accordance with an example of the invention.

FIGS. 7A-7F schematically represent the skin effect and the proximity effect.

FIGS. 8 shows circuitry in an improved charging system, in accordance with an example of the invention.

FIGS. 9A-9C show embodiments of a multi-conductor cable.

FIGS. 10A and 10B illustrate Litz wire and a multi-conductor cable, respectively.

### DETAILED DESCRIPTION

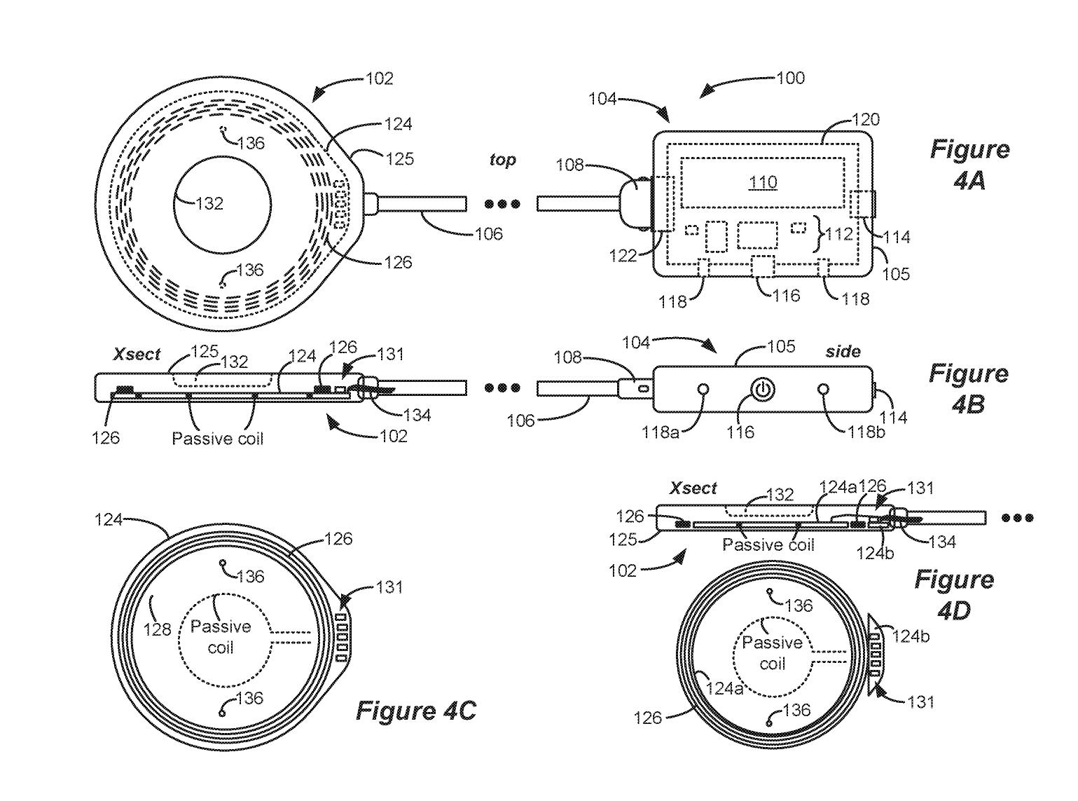

An improved charging system 100 for an IMD 10 is shown in FIG. 4A. Charging system 100 includes two main parts: an electronics module 104 and a charging coil assembly 102 which includes a charging coil 126. The electronics module 104 and the charging coil assembly 102 are connected by a cable 106. The cable 106 may be separable from both the electronics module 104 and the charging coil assembly 102 via a port/connector arrangement. Alternatively, the cable 106 may be permanently or semi-permanently attached to either of or both of the electronics module 104 and/or the charging coil assembly 102. In the illustrated embodiment 100, the cable 106 includes a connector 108 that can attach to and detach from a port 122 of the electronics module 104.

Electronics module 104 preferably includes within its housing 105 a battery 110 and active circuitry 112 needed for charging system operation. Electronics module 104 may further include a port 114 (e.g., a USB port) to allow its battery 110 to be recharged in conventional fashion, and/or to allow data to be read from or programmed into the electronics module, such as new operating software. Housing 105 may also carry a user interface, which as shown in the side view of FIG. 4B can include an on/off switch 116 to begin/terminate generation of the magnetic field 66, and one or more LEDs 118a and 118b. In one example, LED 118a is used to indicate the power status of the electronics module 104. For example, LED 118a may be lit when its battery 110 is charged, and may blink to indicate that the battery 110 needs charging. More complicated user interfaces, such as those incorporating a speaker and a display, could also be used. User interface elements can be included on other faces of the electronic module's housing 105, and may be placed such that they are easily viewed for the therapeutic application at hand (e.g., SCS, DBS). Electronics are integrated within the housing 105 of the electronics module 104 by a circuit board 120.

Charging coil assembly 102 preferably contains only electronic components that are stimulated or read by active circuitry 112 within the electronics module 104. Such components can include the primary charging coil 126 already mentioned, which as illustrated comprises a winding of copper wire and is energized by charging circuitry in the electronics module 104 to create the magnetic charging field 66. One or more passive coils can be included within the charging coil assembly 102, which are used to determine the position and/or alignment of the charging coil 126 (charging coil assembly 102) with respect to the IMD 10 being charged, and more specifically whether the charging coil 126 is aligned and/or centered with respect to an IMD 10 being charged. Additionally, or alternatively, the charging coil assembly 102 may contain one or more coils for sending and receiving telemetry to/from the IMD 10. In the embodiment shown in FIG. 4B, the one or more passive coils are formed using one or more traces in a circuit board 124, which circuit board 124 is also used to integrate the electronic components within the charging coil assembly 102. Circuit board 124 is shown in isolation in FIG. 4C. While in some embodiments the charging coil 126 comprises a wire winding and the one or more passive coils comprise traces within the circuit board 124, this is not strictly necessary: the charging coil 126 can also be formed from circuit board traces, and the one or more passive coils can comprise wire windings. Note that the charging coil 126 and the one or more passive coils, as well as being concentric, are also formed in planes that are parallel and can also be formed in the same plane.

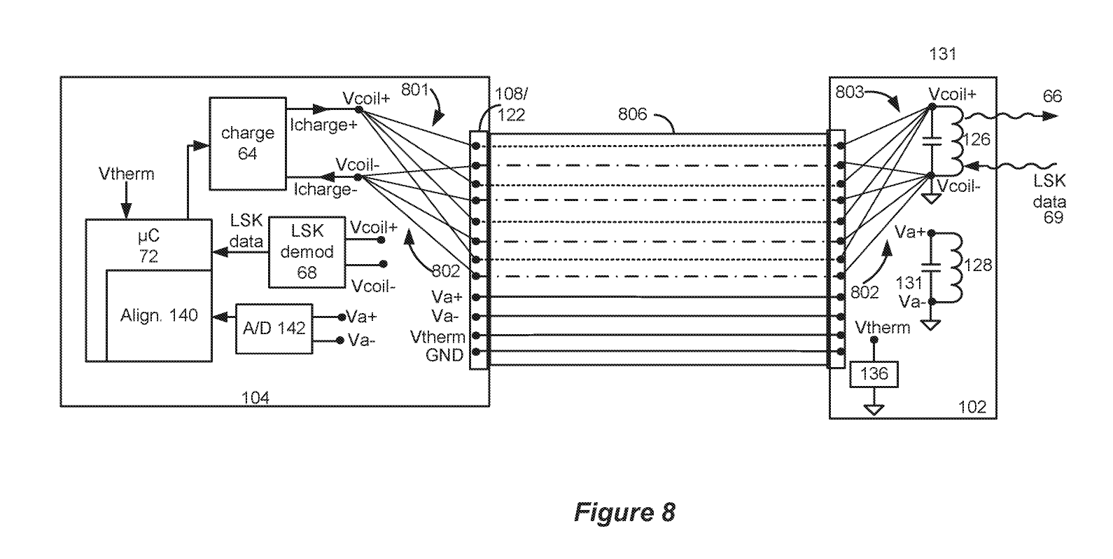

Further passive components preferably included within the charging coil assembly 102 include tuning capacitors 131 coupled to the primary coil 126 and to each one or more passive coils, which is used to generally tune each coils' resonance to that of the magnetic field 66. One skilled in the art will understand that the value of the capacitor 131 (C) connected to the charging coil 126 and to each sense coil will be chosen depending on the inductance (L) of that coil, in accordance with the equation fres=1/sqrt(2πLC). The charging coil assembly 102 can further include one or more thermistors 136, which can be used to report the temperature of the charging coil assembly 102 to the electronics module 104 (FIG. 8, Vtherm). Such temperature data can in turn control production of the magnetic field 66 such that the temperature remains within safe limits. See, e.g., U.S. Pat. No. 8,321,029, describing temperature control in an external charging device.

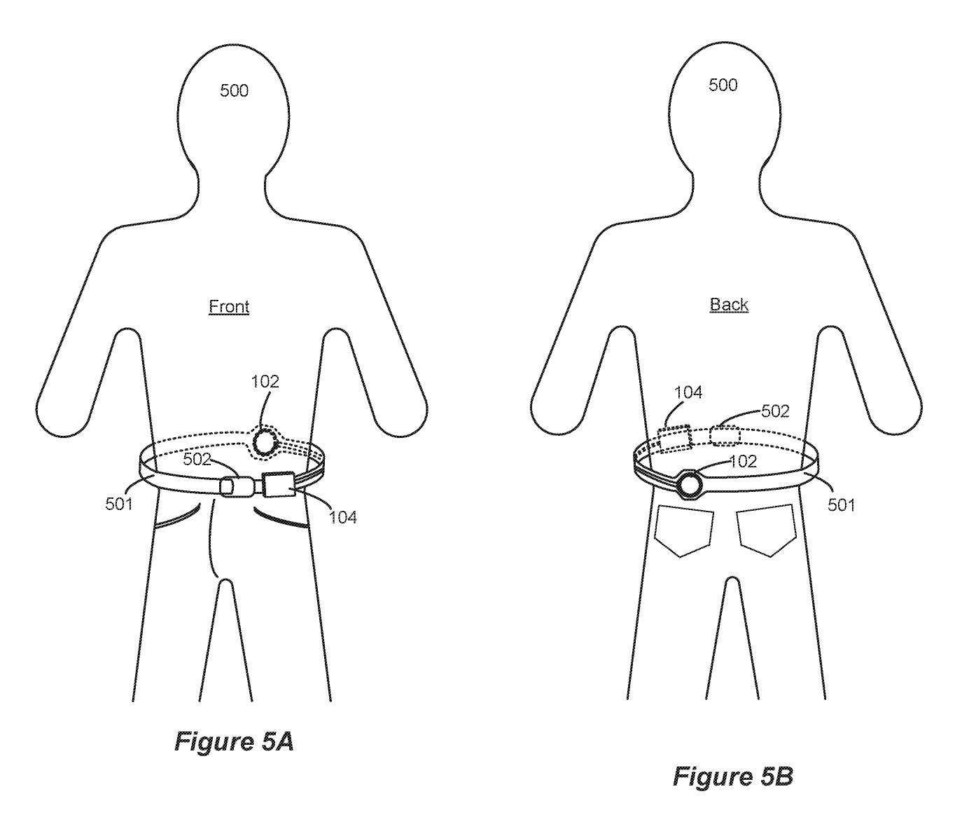

Electronic components within the charging coil assembly 102 can be integrated differently. In FIGS. 4B and 4C, a single circuit board 124 is used, with the charging coil 126 mounted to the patient-facing side of the circuit board 124, and with wires 134 in the cable 106 preferably coupled to the circuit board 124. In FIG. 4D however, two circuit boards 124a and 124b are used. Circuit board 124b is outside of the area of the charging coil 126, and includes the capacitors 131. Circuit board 124a is within the area of the charging coil 126, and includes the one or more passive coils and the thermistors 136. In the two-circuit-board 124a and 124b arrangement of FIG. 5D, notice in the cross section that the charging coil 126 and circuit boards 124a and 124b can be generally located in the same plane, which allows for a thinner construction of the charging coil assembly 102. In FIG. 4D, the wires 134 within the cable 106 can connect to both circuit boards 124a and 124b to allow communication between the components and the electronics module 104. The two circuit boards 124a and 124b can also have connections between them (not shown).

Components in the charging coil assembly 102 are integrated within a housing 125, which may be formed in different ways. In one example, the housing 125 may include top and bottom portions formed of hard plastic that can be screwed, snap fit, ultrasonic welded, or solvent bonded together. Alternatively, housing 125 may include one or more plastic materials that are molded over the electronics components. One side of the housing 125 may include an indentation 132 to accommodate the thickness of a material (not shown) that can be useful to affixing the charging coil assembly 102 to the patient, to the patient's clothes, or within a holding device such as a charging belt or harness. Such material may include Velcro or double-sided tape for example.

FIGS. 5A and 5B show front and back views, respectively, of a patient 500 using a holding device in the form of a belt 501 to hold the electronics module 104 and the charging coil assembly 102 during a charging session. The belt 501 fastens around the patient's waist, and can be secured by a fastening device 502, such as a buckle, clasp, snaps, Velcro, etc. The belt 501 can be adjustable to fit patients with different waist sizes. The belt 501 can include a pouch generally located on the belt 501 in a position where the IMD 10 is implanted in the patient, such as the back of the patient proximate to the buttocks in an SCS application. Alternatively, the charging coil assembly 102 can be held in place by clips, Velcro, etc. Affixing the electronics module 104 and the charging coil assembly 102 to the patient allows the patient to move or walk during a charging session and thus allows charging “on the go.”

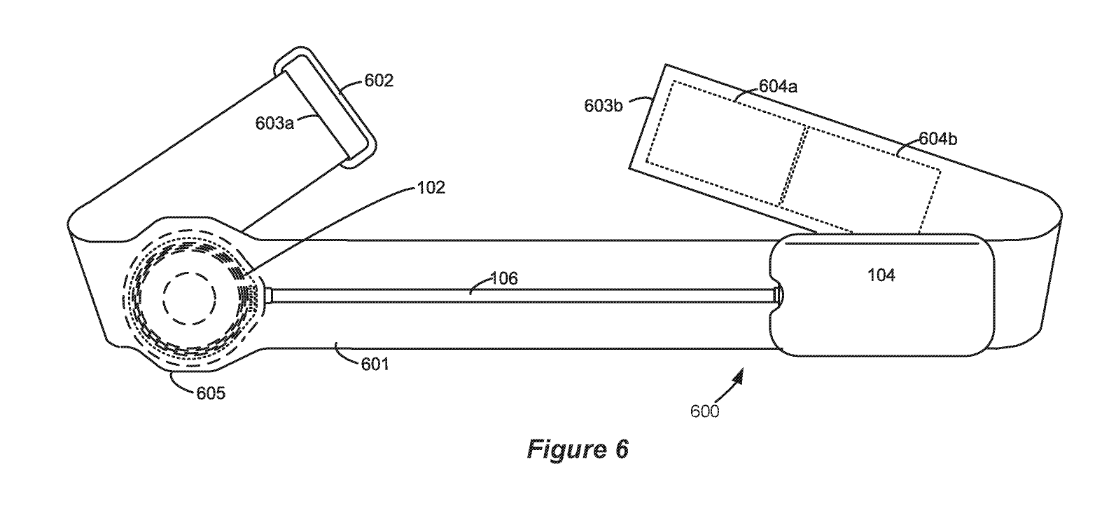

FIG. 6 shows an example of a holding device 600. In the example shown, the holding device comprises a belt 601 that may be worn by an IMD patient. The belt 601 may be formed of cloth such as nylon for example. As shown, belt 601 includes a loop 602 at first end 603a through which the second end 603b of the belt may be passed to fasten the belt 601 to the patient. The second end 603b may include opposing Velcro surfaces 604a and 604b to allow the second end to be secured after passage through the loop. Such fastening means are just one example, and many other well-known fastening means (e.g., buckles, clasps, snaps, hooks, etc.) could be used as well.

Additionally, the holding device 600 need not be fastenable at its ends 603a and 603b to form a closed loop. Instead, the ends 603a and 603b may remain unconnected while still wearable by the patient. This is particularly useful if the holding device 600 comprises a collar draped around a patient's neck, as is useful in a Deep Brain Stimulation (DBS) application for example. The holding device 600 may alternatively be wearable by being affixable to the patient or his clothing by an adhesive for example.

The charging coil assembly 102 can be integrated within the belt 601. Such integration of the charging coil assembly 102 may be effectively permanent, with the assembly 102 stitched between the inner and outer pieces of belt cloth. Alternatively, the belt 601 may be formed of a rubberized material and molded around the charging coil assembly 102. Integration may also be semi-permanent, in which the charging coil assembly 102 is insertable within the belt 601 and thereafter largely left there, although also removeable from time to time (such as to wash the belt, or to switch out the charging coil assembly 102). In this regard, the belt 601 can include a slot into which the charging coil assembly 102 can be inserted between the inner and outer pieces of belt cloth. Such a slot may be openable and closeable, and may include a Velcro flap in one example. The belt 601 may include a flared portion 605 if the charging coil assembly 102 is larger than the width of the belt. Still alternatively, the charging coil assembly 102 may be removeably affixed to the belt 601 with clips, adhesive, Velcro, etc. The electronics module 104 can also be attached to the belt 601 with clips, adhesive, Velcro, etc. The cable 106 connecting the electronics module 104 to the charging coil assembly may be permanently or semi-permanently integrated into the belt 601 using any of the options described above for integrating the charging coil assembly into the belt. Alternatively, the cable 106 may be removeably attached to the belt by running the cable through loops in the belt, or using, hooks, clips, etc.

As shown in FIGS. 5 and 6, the cable 106 connecting the electronics module 104 and the charging coil assembly 102 must be long enough to wrap partially around a patient's body. Thus, the cable 106 may be on the order of a meter in length in some instances. A cable of substantial length may give rise to some difficulties related to powering a coil, such as charging coil 126, for reasons explained here.

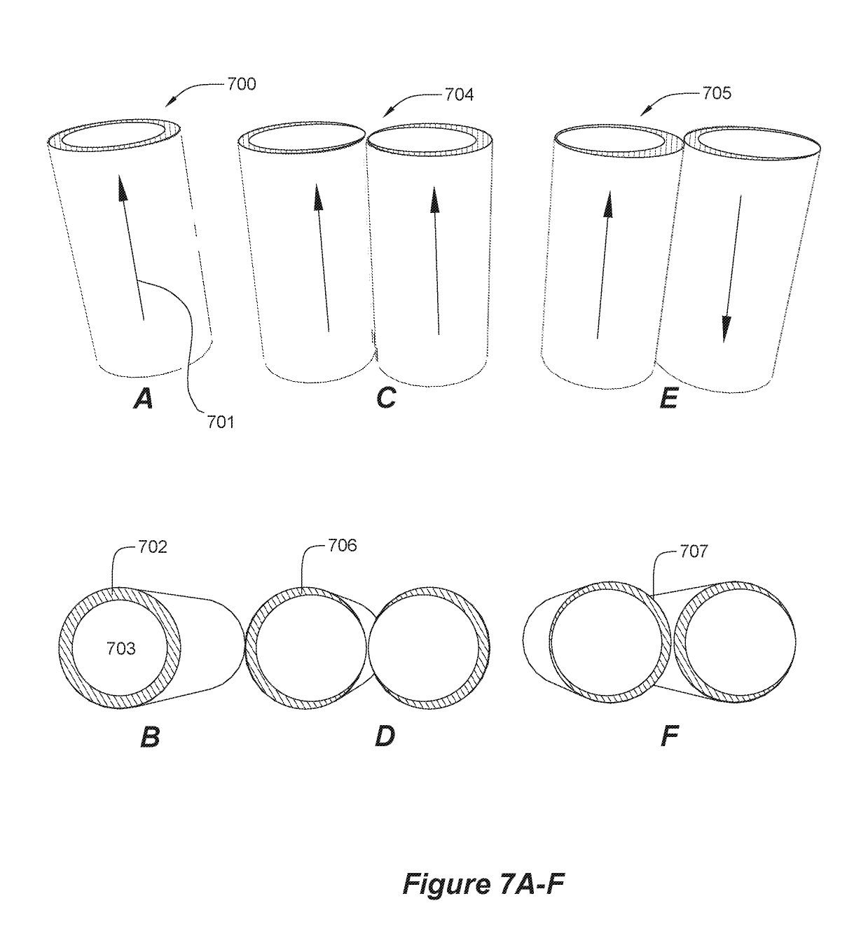

The electronics module 104 typically powers the charging coil 126 by providing an AC current to the coil 126 via conductors in the cable 106. The frequency of the AC current is generally on the order of about 80 KHz, a frequency that generates magnetic fields that can efficiently penetrate a patient's tissue and transcutaneously charge an IMD 10. A person of skill in the art will appreciate that a conductor carrying an AC current having a sufficiently high frequency exhibits a physical phenomenon referred to as “skin effect.” The skin effect is illustrated in FIGS. 7A and 7B, which shows a section of a conductor 700 in longitudinal view (FIG. 7A) and cross sectional view (FIG. 7B). In a conductor 700 carrying a high frequency AC current 701, most of the current becomes preferentially distributed near the surface of the conductor, illustrated by the shaded region 702. The interior portion 703 of the conductor carries much less current. As a result, the current is carried by a smaller effective cross section of conductor, effectively increasing the resistance of the conductor.

A potential improvement for addressing the skin effect is to use stranded conductor, i.e., conductor having multiple strands instead of a single strand. For a given cross sectional area of conductor, the ratio of surface to interior area is greater for a stranded conductor compared to a single strand. However, stranded cable gives rise to another effect, referred to as “proximity effect.” The proximity effect is illustrated in FIGS. 7C-7F, which shows two pairs of conductors 704 and 705 carrying AC current of the same polarity (FIGS. 7C and 7D) and of the opposite polarity (FIGS. 7E and 7F), respectively. The alternating magnetic field caused by the AC current in one conductor creates eddy currents in the neighboring conductor(s), which warp the AC current in that conductor. When the proximate conductors carry current having the same polarity, as shown in FIGS. 7C and 7D, the eddy currents cause the AC currents to become preferentially distributed on the surface of the conductors away from each other, as illustrated by the shaded area 706. In other words, the AC current is not only preferentially confined to the surface of the conductor; it is also preferentially confined to an area of the surface that is distant from neighboring AC carrying conductors. As illustrated in FIGS. 7E and 7F, when proximate conductors carry AC current of opposite polarity, the proximity effect causes the current to preferentially concentrate in areas of the surface 707 nearest to each other. Both the proximity effect and the skin effect constrain the current to a small region of the conductors, thereby increasing the resistance of the conductors. That increase in resistance can cause a large portion of the power delivered by the electronics module 104 to be lost in the cable 106 and therefore not delivered to the charging coil 126.

One method of reducing the resistance of the such a cable is by using Litz wire for carrying current between the coil and the electronics module. Litz wire utilizes many thin, individually insulated wire strands woven or twisted together in a prescribed pattern designed to cancel the proximity effect. However, implementing Litz wire in a cable such as cable 106, which typically contains multiple conductors for in addition to the power conductors, can be difficult and requires a substantial amount of customization as well as customized cable terminations or connectors.

The inventors have found that a multi-conductor cable, such as a standard ribbon cable, can be used as cable 106 in a configuration that achieves some of the same benefits as Litz wire but without custom cable and connector requirements. FIG. 8 shows a schematic diagram of the electronics module 104, the charging coil assembly 102, and a ribbon cable 806 (implemented as cable 106, as illustrated in FIG. 4). The electronics module 104 may include (as part of circuitry 112; FIG. 4A) control circuitry 72 that controls charging circuitry 64 to generate an AC charging current. The charging circuitry 64 includes two terminals Vcharge+ and Vcharge−, which supply current Icharge+ and Icharge−, respectively, to the charging coil 126. The polarity of Vcharge+/− and Icharge+/− alternate at a frequency that is typically on the order of 80 KHz. A plurality of traces 801 in the circuit board 120 connect the Vcharge+ to connectors at terminal port 122. Four traces 801 are shown in FIG. 8 but the number of traces can vary. Likewise, a plurality of traces 802 connect the Vcharge− terminal to connectors at port 122. The Icharge+ current is carried on a plurality of conductors represented as dashes (---) within cable 106. The Icharge− current is carried on a plurality of conductors represented as dash-dots (-.-). Complimentary pluralities of traces 803 and 804 within the charging coil assembly 102 conduct the Icharge+/− current to the coil 126, providing voltage Vcoil+/− across the coil, energizing the charging coil to produce the magnetic field 66. Using multiple small, individually insulated conductors within ribbon cable 806 instead of one large conductor to carry the Icharge+/− currents decreases the resistance of the of the cable 806 by minimizing the skin effect and spacing the conductors apart from each other minimizes the resistance due to the proximity effect and the skin effect.

The cable 806 must have an adequate number of discrete conductors to carry the AC powering current and any other AC or DC signals required for a particular electronics module/charging coil assembly pair. For example, the electronics module 104 illustrated in FIG. 8 includes an LSK demodulator 68 that can demodulate LSK data 69 encoded upon the reflected impedance of the charging coil 126. Two terminals, Vcoil+ and Vcoil, provide the input to the LSK Demodulator 68. Those two terminals may be connected (directly or indirectly) to the pluralities of traces 801 and 802 or they may be connected to other pluralities of traces with complementary pluralities of independent conductors within cable 806.

The charging coil assembly 102 illustrated in FIG. 8 also includes a passive coil 128, which, in that embodiment, is an alignment coil. Examples of alignment coils are described, for example, in commonly owned U.S. Patent Application Ser. No. 62/350,451, filed Jun. 15, 2016. The passive coil 128 includes terminals Va+ and Va−, which connect to corresponding terminals Va+ and Va− of an analog to digital converter 142 of the electronics module 104. The output of the analog to digital converter 142 is provided to an alignment circuit 140, as described in the 'XXX application. The illustrated cable 106 includes single conductors for each of Va+ and Va−. However, multiple discrete conductors could be used for the passive coil 128 in a manner similar to the multiple conductors used for the charging coil 126. A plurality of traces would connect each of Va+ and Va− to port 122.

The illustrated charging coil assembly 102 also includes a thermistor, which generates a DC voltage Vtherm as a function of temperature. Vtherm is provided to the microcontroller 72, which may be programmed to adjust charging based on the measured temperature. Typically, only a single conductor, as included in the illustrated cable 106, is needed to communicate Vtherm between the charging coil assembly 102 and the electronics module 104 because Vtherm is a DC voltage. The illustrated cable 806 also includes a single conductor GND providing a ground between the electronics module 104 and the charging coil assembly 102.

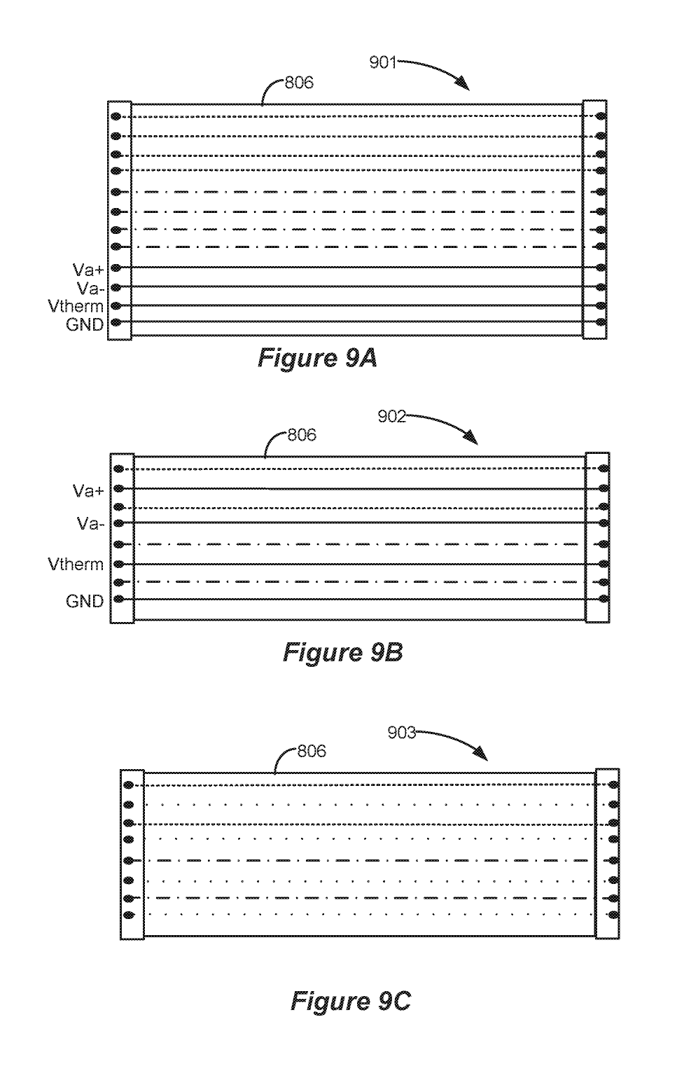

In the illustrated cable 806 of FIG. 8, the DC conductors (solid lines) are grouped together in one part of the cable 806 and the AC Icharge+ and Icharge− conductors are interspersed with each other in a different part of the cable. However, many different configurations are possible. FIGS. 9A, B, and C show three alternative configurations, 901, 901 and 903, respectively. For clarity, only the cable 806 is shown. In configuration 901 the Icharge+ conductors (---) are grouped together and the Icharge− conductors (-.-) are grouped together. In configuration 902, the DC conductors Va+, Va−, Vtherm, and GND are interspersed between the AC conductors Icharge+ and Icharge− (note that only four AC conductors are shown, but the number can vary). In configuration 903, grounded dummy conductors (represented as dotted lines . . . ) are interspersed between the AC conductors Icharge+ and Icharge−. Many other configurations are possible.

An advantage of the illustrated cable 806 is that each of the conductors are physically the same. In other words, it need not be the case that some conductors are made of Litz wire, some of single stranded wire, some of stranded wire, etc. Thus, the cable need not be customized for each particular configuration of an electronics module and/or charging coil assembly, so long as the cable has an adequate number of conductors. Numerous multi-conductor cables are available, such as ribbon cable, serial cable, USB cable, and the like. For example, ribbon cable having 4, 6, 8, 9, 10, 14, 15, 16, 18, 20, and up to about 80 conductors are available, each with standard connectors. Likewise, serial cables having 4, 9, 25 and other numbers of conductors, with standard connectors. Moreover, according to some embodiments, the cable 106 can be permanently attached (i.e., hardwired) to one or both the electronics module 104 and/or the charging coil assembly 102. In such a case, the conductors of the cable may be soldered to soldering pads on the PCB.

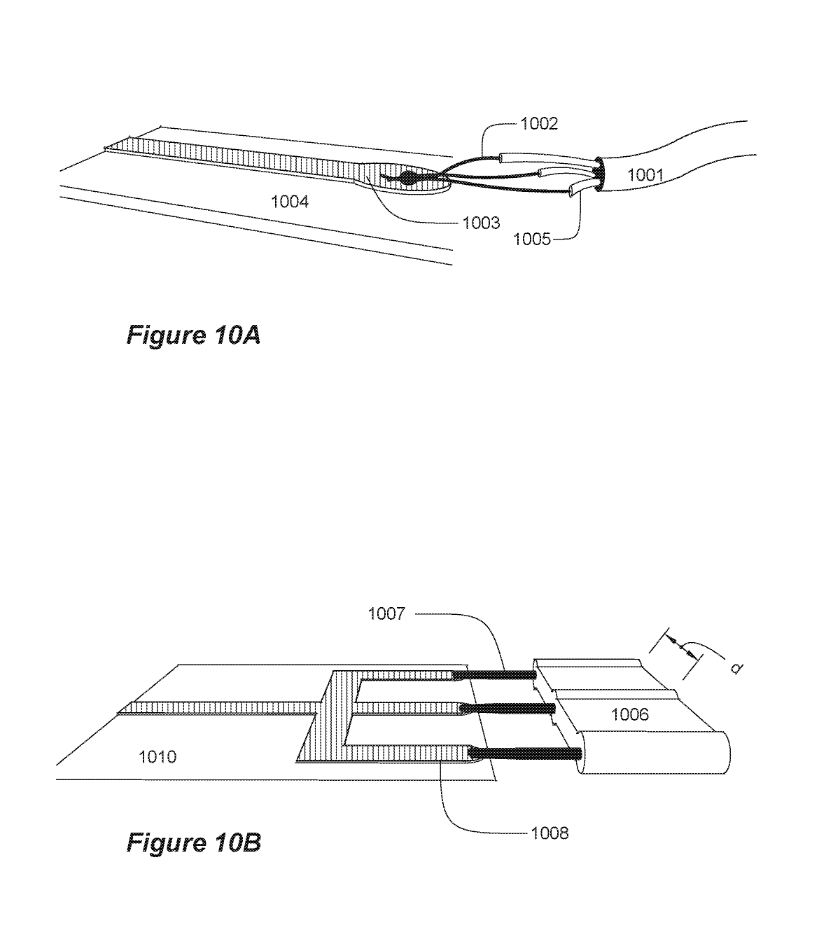

FIGS. 10A and 10B illustrate how using multiple individually insulated conductors to carry AC current, as described above, differs from simply using Litz wire. FIG. 10A shows the conductors 1002 of a Litz wire 1001 bonded to a bonding pad 1003 of a PCB 1004. Even though the conductors 1002 of the Litz wire 1001 are individually insulated with insulation 1005, they are still held intimately close together within the Litz wire 1001. They are therefore susceptible to the proximity effect. By contrast, the multi-conductor cable 1006 maintains separation d of conductors 1007. Each of the conductors are connected to separate connectors 1008 (solder pads in the illustration) on PCB 1010.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover equivalents that may fall within the spirit and scope of the present invention as defined by the claims.

What is claimed is:

1. A charger for wirelessly providing power to an implantable medical device (IMD), the external charger comprising:
an electronics module, a charging coil assembly, and a cable configured to connect the electronics module with the charging coil assembly; wherein
the cable comprises multiple individually insulated conductors; and
the electronics module comprises driving circuitry configured to provide AC current to the charging coil assembly, the driving circuitry having a first terminal having a first polarity and a second terminal having a second polarity, wherein the first terminal is connected to a first plurality of connectors for connecting with the cable and the second terminal is connected to a second plurality of connectors for connecting with the cable; wherein
the electronics module is configured so that when the cable is connected to the electronics module each of the first plurality of connectors contacts one or more of the first plurality of individually insulated conductors and each of the second plurality of connectors contacts one or more of the second plurality of conductors.

2. The charger of claim 1, wherein the cable is a ribbon cable.

3. The charger of claim 1, wherein the cable is a serial cable.

4. The charger of claim 1, wherein the cable is a USB cable.

5. The charger of claim 1, wherein the cable permanently connects to the electronics module.

6. The charger of claim 1, wherein the first plurality of connectors and the second plurality of connectors are bonding pads.

7. The charger of claim 1, wherein the first and second pluralities of connectors are configured to connect to an insulation displacement contact (IDC) of the cable.

8. The charger of claim 1, wherein the first and second pluralities of connectors comprise pins.

9. The charger of claim 1, wherein the first plurality of connectors and the second plurality of connectors are comprised within a port.

10. The charger of claim 9, wherein the port is a USB port. The charger of claim 9, wherein the port is a serial port.

12. The charger of claim 9, wherein the port is a parallel port.

13. A charger for charging an implantable medical device (IMD), the charger comprising:
a printed circuit board (PCB);
circuitry connected to the PCB for providing AC current, the circuitry comprising a first terminal having a first polarity and a second terminal having a second polarity;
a first plurality of conductive traces on the PCB connecting the first terminal to a first plurality of connectors; and
a second plurality of conductive traces on the PCB connecting the second terminal to a second plurality of traces.

14. The charger of claim 13, wherein the first and second connectors are pins.

15. The charger of claim 13, wherein the first and second connectors are bonding pads.

16. The charger of claim 13, wherein the first and second connectors are comprised within a port.

17. The charger of claim 16, wherein the port is serial port.

18. The charger of claim 16, wherein the port is a parallel port.

19. The charger of claim 16, wherein in port is a USB port.
